(19) United States
(12) Patent Application Publication
(10) Pub. No.: US 2021/0261891 A1
(43) Pub. Date: Aug. 26, 2021

(54) Method For Preparing Surfactant-Containing Compositions In A Sequential Process

(71) Applicant: Henkel AG & Co. KGaA, Duesseldorf (DE)

(72) Inventors: Gerd Boesemann, Duesseldorf (DE); Matthias Sunder, Duesseldorf (DE); Volker Blank, Leverkusen (DE)

(21) Appl. No.: 17/032,430

(22) Filed: Sep. 25, 2020

(30) Foreign Application Priority Data
Sep. 27, 2019 (DE) 102019126124.4

(51) Int. Cl. C11D 17/04; A61Q 19/10; A61K 8/96; C11D 11/00; C11D 3/42; C11D 3/386

(52) U.S. Cl. CPC C11D 17/045; A61Q 19/10; A61K 8/96; C11D 11/0017; A61K 2800/87; C11D 3/386; C11D 17/046; C11D 11/0094; A61K 2800/88; C11D 3/42

(57) ABSTRACT

The present invention relates to a method for preparing liquid surfactant-containing compositions in a sequential, continuous process which makes it possible to fill multi-chamber containers simultaneously with different compositions.

### FIELD OF THE INVENTION

The present invention relates to a method for preparing liquid surfactant-containing compositions in a sequential, continuous process which makes it possible to fill multi-chamber containers simultaneously with different compositions.

### BACKGROUND OF THE INVENTION

Liquid, surfactant-containing compositions have become an integral part of everyday life. These compositions can be body care products, such as shampoos, shower gels or bubble baths. However, they also include washing or cleaning agents, such as household cleaners, softeners, detergents for laundry, floor care products, all-purpose cleaners, manual dishwashing detergents, automatic dishwasher detergents or heavy-duty detergents.

Today, most of these compositions are prepared in a batch process. The batch process, also often referred to as batch production, is a discontinuous production process. In this case, specific amounts of feed materials are conveyed into a container according to a predetermined formula and mixed there. The capacity of the production vessel in which all the constituents are mixed together limits the amount of material that can be prepared in a batch.

In a typical batch process, a reaction vessel is first completely filled with the starting materials. The reaction of the starting materials with one another to form the end product takes place within the reaction vessel. When the reaction that has optionally taken place is completed, the reaction vessel is completely emptied and the desired formulation is filled into suitable containers for sale or optionally for storage. The reaction vessel must then be prepared for the next filling. This means that, if another formulation is prepared, the reaction vessel and optionally the lines through which the starting products are introduced into the reaction vessel are thoroughly cleaned.

Such a batch process has the advantage that the formulation of the formula can still be adjusted in the reaction vessel if necessary. Subsequent metering of individual components is possible here. In terms of quality, it must be taken into account that it is possible to trace batches.

However, the large amount of space required is disadvantageous. A reaction vessel is always completely filled, i.e. large quantities of a product are always prepared. When a batch has been prepared, it must first be processed before another batch can be started. If direct processing or filling is not possible, a product that has already been prepared must be stored outside the reaction vessel. This leads again to a high space requirement and additional costs.

Furthermore, the change in production from one product to another requires a lot of effort. If, for example, a product that has a specific dye and a specific odorant is prepared in a first batch process, the reaction vessel and all supply lines must be cleaned thoroughly before a second product having a different color and odor profile is prepared, so as to avoid contaminating the batches.

In addition to the discontinuous batch process, continuous processes for the production of liquid, surfactant-containing compositions are also known. Continuous processes offer better opportunities for just-in-time production. However, complex control of the individual process steps is necessary in this case. In the continuous process, mixing takes place by means of static or dynamic mixing devices and not in a reaction vessel as in the batch process. Rather, the mixing takes place within a line. The individual ingredients of a formula are metered into this line in a predefined sequence. Filling takes place immediately at the end of this line. It is not possible to subsequently meter or change the concentration of individual constituents in this case. Targeted and controlled monitoring of the addition of each individual constituent is necessary.

When preparing body care, washing or cleaning agents, it should also be noted that the addition of solid constitution may be necessary. However, these can be added only in a batch process. The addition of solid constituents in a continuous process is not possible or is possible only with great difficulty. In a continuous process, only liquid components can be metered. Solid constituents must be in the form of a suspension in order to be metered. However, this causes inaccuracies in the concentration.

However, the addition of solid additives to corresponding compositions is currently part of the prior art. Stably suspending solids in liquids is often problematic, in particular if the solids differ in density from the liquid; they tend to sediment or float. Incorporating certain active ingredients (e.g. bleaching agents, enzymes, perfumes, dyes, etc.) into liquid washing and cleaning agents can also lead to problems. For example, there may be incompatibilities between the individual active-ingredient components of the liquid washing and cleaning agents. This can lead to undesirable discoloration, agglomeration, odor problems and destruction of washing-active active ingredients.

Methods for preparing liquid compositions that address this problem are described, for example, in WO 2016/091733 A1 or WO 2017/001218 A1.

### BRIEF SUMMARY OF THE INVENTION

In order to avoid incompatibilities between active-ingredient components, liquid washing and cleaning agents are increasingly being offered in multi-chamber containers. Different compositions are provided in what are referred to as pouches or bags in two or more chambers. This makes it possible to prevent interactions between individual components, as these components are present in separate chambers. The compositions are then released during use. This allows longer storage and a greater possibility of compositions since incompatibilities between the individual active-ingredient components can be dealt with by separate provision.

However, the provision in multi-chamber bags requires that the different compositions are provided simultaneously in order to then fill them into the respective chambers of the multi-chamber bags. One possibility is to prepare the different compositions separately in a batch process. Alternatively, it would be possible to use methods, as described in WO 2017/001218 A1 or WO 2016/091733 A1, to prepare the individual compositions. In both cases, each composition would then have to be prepared and stored until all of the compositions have been prepared. Filling subsequently takes place. In a method as described in WO 2017/001218 A1, two or more methods could be carried out in parallel in order to prepare the individual compositions. However, these options require a redundant installation of all components and, as a result, require a large amount of space. In addition, the required amounts of the individual compositions are small, since the amount per composition in a chamber is small. Correspondingly, a system would not be used optimally, and therefore there is also a need for an economical solution.

It has surprisingly been found that the disadvantages arising from the prior art can be substantially prevented by a method for the continuous preparation of at least two different liquid, surfactant-containing compositions. In a further embodiment, the invention relates to multi-chamber bags having at least two separate chambers and to a method for cleaning textile structures wherein the structure to be cleaned is placed in a drum of a washing machine with a multi-chamber bag and an automated washing program of the washing machine is started.

In a first embodiment, the present invention therefore relates to a method for the continuous preparation of at least two different liquid, surfactant-containing compositions, in which method a flow of a master batch which has at least one surfactant and a solvent is continuously provided. This master batch cyclically goes through at least the following steps:
a. adding at least one first additive to the flow of the master batch for a first length of time in order to obtain a first composition,
   and
b. adding at least one second additive to the flow of the master batch for a second length of time in order to obtain a second composition,

the at least one first additive and the at least one second additive being different from one another and the first and second compositions being stored separately.

According to the invention, storage takes place only for a very short period of time, which ensures that sufficient quantities of the first composition and the second composition are present in order to fill these compositions simultaneously into separate chambers of a multi-chamber container. Surprisingly, it has been shown that a corresponding sequential preparation method of at least two different compositions makes it possible to provide, with high precision, compositions which can be filled into a multi-chamber bag simultaneously without there being a need for space for systems operated in parallel.

It would also be conceivable to use a device as described in WO 2017/001218 A1 and to separate the final flow into 2, 3 or more flows. However, it has been shown that such a variant with a splitter cannot be implemented technically in such a way that three partial flows of the same size are obtained, so that fluctuations in the formulation of the individual compositions cannot be prevented. However, this should be avoided since a constant composition is necessary in order to ensure the same cleaning performance for each multi-chamber bag.

The method according to the invention is therefore a continuous preparation method for two or more different liquid surfactant-containing compositions, the compositions differing in terms of at least one component. Correspondingly, a master batch is first provided, which includes the constituents that are included in both compositions, such as surfactant and solvent.

The compositions are then obtained by adding a first additive to the master batch to obtain a first composition and adding a second additive to obtain a second composition. The at least one first additive and the at least one second additive are different from one another. Both the at least one first additive and the at least one second additive comprise at least one dye. In this case, the dyes are different from one another, such that the compositions differ from one another at least in terms of their color. Suitable dyes by means of which a liquid surfactant-containing composition can be colored without this being disadvantageous in use and, for example, leading to discoloration of textiles or other surfaces with which the compositions come into contact are well known to the person skilled in the art.

Preferably, not only two, but three, four or more different compositions are prepared in the method according to the invention. Correspondingly, the master batch cyclically goes through at least the following steps in the case of three different compositions:
a. adding at least one first additive to the flow of the master batch for a first length of time in order to obtain a first composition, and
b. adding at least one second additive to the flow of the master batch for a second length of time in order to obtain a second composition,
c. adding at least one third additive to the flow of the master batch for a third length of time in order to obtain a third composition,
   the at least one first additive and the at least one second additive and the at least one third additive being different from one another.

Correspondingly, in such a method the at least one first additive and the at least one second additive and the at least one third additive are different from one another.

Similarly, the master batch cyclically goes through at least the following steps in the event that four different liquid, surfactant-containing compositions are prepared:
a. adding at least one first additive to the flow of the master batch for a first length of time in order to obtain a first composition, and
b. adding at least one second additive to the flow of the master batch for a second length of time in order to obtain a second composition,
c. adding at least one third additive to the flow of the master batch for a third length of time in order to obtain a third composition,
d. adding at least one fourth additive to the flow of the master batch for a fourth length of time in order to obtain a fourth composition.

In this case, too, the at least one first additive, the at least one second additive and the at least one third additive, as well as the at least one fourth additive, are different from one another.

First, second, optionally third and optionally fourth and any further lengths of time can be the same or different. The length of time can also change. If, for example, the master batch cyclically goes through steps A and B, the first length of time can be t1 when step A is run through for the first time, and the second length of time for step B is t2. When the steps are repeated cyclically, the first length of time would then be t3 and the second length of time t4. Further cyclical repetitions are possible. The lengths of time t1 and t3 may be different from one another, for example. They can also be different from one another. t3 can be longer or shorter than t1. In a renewed cyclical run, the first length of time would be t5 and the second length of time t6. For example, t3 may be greater than t1 and t5 could correspond to t1. It is also possible for t5 to be smaller than t3 but larger than t1. t5 could also be less than t1 or greater than t3. It would also be possible for t5 to correspond to t3.

All variations are also possible for the second lengths of time t2, t4 and t6. Setting the length of time depends on the amount of composition that is prepared in order to fill individual compartments of multi-compartment bags. The size of the chambers can vary, and therefore the required amount of compositions that are provided varies. A person skilled in the art is able to optimize the lengths of time in order to optimize the intermediate storage before filling.

Of course, the same applies not only to the first and second lengths of time, but also to the third and each further length of time, depending on how many different compositions are provided.

The at least one first additive and/or the at least one second additive and/or optionally the at least one third and each further additive preferably have, in addition to the dye, at least one or more enzymes, the enzymes particularly preferably being different from one another.

It is also possible for the at least one first additive and/or the at least one second additive and/or the optionally present at least one third additive and/or each optionally further present additive to comprise at least one optical brightener. If an additive comprises an optical brightener, said additive does not include an enzyme. Preferably, at least one of the additives comprises at least one enzyme and at least one of the additives comprises at least one optical brightener. Optical brighteners (so-called “whiteners”) can be added to the liquid washing and cleaning agents in order to remove graying and yellowing of the treated textile fabrics. These substances are absorbed by the fiber and have a brightening and simulated bleaching effect by converting invisible ultraviolet radiation into visible longer-wave light, the ultraviolet light absorbed from the sunlight being emitted as a slightly bluish fluorescence and, together with the yellow tone of the grayed or yellowed laundry, produces pure white. Within the meaning of the present invention, suitable compounds originate, for example, from the substance classes of 4,4′-diamino-2,2′-stilbenedisulfonic acids (flavonic acids), 4,4′-distyryl-biphenyls, methylumbelliferones, coumarins, dihydroquinolinones, 1,3-diarylpyrazolines, naphthalic acid imides, benzoxazole, benzisoxazole and benzimidazole systems as well as the pyrene derivatives substituted by heterocycles. The optical brighteners are preferably used in amounts between 0.03 and 0.3 wt. % based on the finished agent.

The master batch, which comprises at least one surfactant and at least one solvent, can be prepared in a continuous process or a batch process. The master batch is preferably prepared by providing a premixture comprising at least one surfactant and at least one solvent being prepared, to which premixture at least one further component is then added in a continuous process. This one further component can be, for example, perfumes, solvents, co-surfactants, preservatives, color-protection agents, bleach inhibitors, pH adjusters, structural aids, corrosion inhibitors and others. These components are selected such that they are included in all of the compositions prepared.

### BRIEF DESCRIPTION OF THE DRAWINGS

FIG. 1 shows a flow chart of an embodiment of the method of the invention

### DETAILED DESCRIPTION OF THE INVENTION

In the preferred method, in which a premixture is prepared in a batch process, to which premixture at least one further component is subsequently added in a continuous process, the proportion of all the constituents of the mixture prepared in the batch process is 1 wt. % to 99 wt. %, preferably 5 wt. % to 95 wt. %, in particular 20 wt. % to 90 wt. %, based on the total weight of the master batch. The proportion of all the constituents that are introduced in the continuous process is preferably 1 wt. % to 99 wt. %, in particular 5 wt. % to 95 wt. %, more preferably 10 wt. % to 80 wt. %.

In this preferred method, the premixture has a temperature in the range of 35° C. or more. This means that the premixture, which is fed from the batch boiler and then further processed in a continuous system, has a temperature of 35° C. or more when it enters the continuous system. The temperature of the mixture is determined in the batch boiler and in the continuous system on the supply line by means of a commercially available PT100 resistance thermometer. The thermometer is installed in the batch boiler next to the outlet through which the mixture reaches the continuous system. Usually, the mixture in the batch boiler has the same temperature during discharge as at the time of introduction into the continuous system. This is checked by means of a second PT100 resistance thermometer, which is installed in the continuous system at the point at which the mixture is supplied. The mixture is always prevented from cooling below 35° C. between the batch boiler and the introduction into the continuous system. The temperature of the mixture in the batch boiler is optionally set significantly above 35° C. in order to introduce the mixture into the continuous system at 35° C. or more. The mixture is therefore not reheated between the boiler and the continuous system before it reaches the continuous system. Rather, the heat of the batch mixture is used to feed the mixture into the continuous system without further heating at a temperature of 35° C. or more. This is a particular advantage of the present invention because it helps to save energy and stabilize the mixture.

Premixtures are usually prepared in the batch process at an increased temperature. In most processes this is 35° C. or higher. The premixture often has temperatures in the range of from 40° C. to 90° C. at the end of the batch process.

The batch temperature is often based on a solvent having a temperature of 40° C. or more, in particular of 50° C. or more, preferably of 60° C. or more, being used. These temperatures allow the active substances which are to be dissolved in the solvent in the batch process to dissolve well or to be distributed therein. According to the invention, the solvent can be introduced into the batch process at a temperature above room temperature. Within the meaning of the present invention, room temperature means 20° C. In addition to the solvent, other substances that have one of the temperatures described above can also be added to the batch.

However, it is also possible for the entire premixture to be heated in the batch process. This can be carried out by frictional or shear forces that occur during mixing in the batch process. Heating elements can also be used to heat the premixture in the batch boiler. However, exothermic reactions often also take place in the batch process, in which reactions additional heat is released, as a result of which the temperature in the stirred tank of the batch process increases. Corresponding exothermic reactions are, for example, neutralization reactions which occur when surfactants, in particular anionic surfactants, are produced in the batch by neutralization of the corresponding acid when, for example, dilution is carried out by concentrated alkali.

Acids of the anionic surfactants disclosed herein can therefore be neutralized by a suitable neutralizing agent in the batch boiler or outside the batch boiler. The heat generated by the neutralization reaction in the boiler or by the supply of the warm neutralizate in the boiler increases the temperature of the mixture in the batch. This improves the solubility of the individual components in the mixture. Suitable neutralizing agents in the context of the present invention are all substances which are able to neutralize the anionic surfactant in its acid form, i.e. convert it into an anionic surfactant salt.

The neutralizing agent can be added in the liquid or solid state. Neutralizing agents in the liquid state include solutions and suspensions of solid neutralizing agents. For example, alkali metal hydroxides such as NaOH or KOH, basic oxides such as alkali metal oxides or basic salts such as carbonates are suitable.

Other neutralizing agents are ammonia and amines. Amines are preferably selected, in particular from the group consisting of monoethanolamine, trimethylamine, triethylamine, tripropylamine, triethanolamine, N-methylmorpholine, morpholine, 2,2-dimethylmonoethanolamine, N,N-dimethylmonoethanolamine and mixtures thereof. The amines are very particularly preferred since they are easy to handle and no water is formed during the neutralization. Monoethanolamine is particularly preferred.

The neutralizing agents can be combined with the anionic surfactant acids that are conventional for washing agents, cleaning agents and care products, in particular with the anionic surfactant acids corresponding to the anionic surfactants disclosed herein. Neutralizing agents are preferably used in a specific molar stoichiometric ratio to the anionic surfactant acid, which allows the reaction to proceed to completion under the selected reaction conditions. For example, the molar ratio of neutralizing agent to anionic surfactant acid may be 0.5:1 to 10:1, preferably 1:1 to 3:1.

It may be advantageous to heat the anionic surfactant acid and/or the neutralizing agent or the mixture in the batch kettle in order to accelerate the start of the neutralization. A particularly preferred anionic surfactant acid is C9 to C13 alkyl benzene sulfonic acid, in particular linear C9 to C13 alkyl benzene sulfonic acid. In particular, linear C9 to C13 alkyl benzene sulfonic acid (LAS acid or HLAS) and monoethanolamines, which are preferably constituents of the premixture prepared in the batch process or of the master batch, react with one another with the production of heat. In a preferred embodiment of the method according to the invention, linear C9-C13 alkyl benzene sulfonic acid is neutralized with monoethanolamine in the batch process.

A particular advantage of using monoethanolamine is preventing water from forming as a neutralization product. This is significant in particular in the preparation of water-free or low-water mixtures and compositions. It is advantageous to prepare the anionic surfactants from the corresponding acids only in the batch, since the acid is cheaper to buy and the heat of neutralization heats the mixture, so that the dissolution of the components in the premixture is accelerated. In specific embodiments, the further targeted supply of heat can be dispensed with, which allows a more economical process flow. Heat can also be released when one or more active substances are mixed in a solvent. Such a release of heat is preferred in the batch process, since this means that most of the constituents are more soluble in the solvent.

In addition, it is known that important raw materials, such as enzymes, silicones (defoamers), fragrances or solvents having a low flash point can only remain stable or be metered in a liquid mass at temperatures T<30° C. There is also a high probability that at T>30° C. enzymes that may be present in the composition will degrade significantly faster, causing a decrease in product performance. A silicone emulsion included as a defoamer can also break at increased temperatures, causing a phase separation in the product. This can result in the batch being foamed, so that further processing is no longer possible. According to the invention, the mixture prepared in the batch process is therefore preferably free from defoamers. According to the invention, these can be introduced into the composition in the continuous process. Therefore, in one embodiment the premixture can be provided with defoamer in a continuous process, in particular such that the composition comprises at least 0.01 wt. % defoamer. In the batch process, solvents having a low flash point can escape and thus form an explosive atmosphere, which can endanger the safety of the preparation process, and therefore these solvents are also preferably added in a continuous process. The master batch can also comprise defoamer regardless of the preparation method.

Within the meaning of the present invention, enzymes are all known enzymes suitable in washing or cleaning agent methods, for example amylases, lipases, cellulases, pectinase and proteases.

Within the meaning of the present invention, defoamers are silicones. The concentration of the defoamers in the composition is preferably 0.005 to 0.02 wt. %.

Silicone oils are particularly preferred. Suitable silicones are conventional organopolysiloxanes, which can have a content of finely divided silicic acid which in turn can also be silanized. Such organopolysiloxanes are described, for example, in European patent application EP 0496510 A1. Polydiorganosiloxanes which are known from the prior art are particularly preferred. Polydiorganosiloxanes usually include finely divided silicic acid, which can also be silanized. Silica-containing dimethylpolysiloxanes of maleic acid are particularly suitable.

The temperature of the mixture is reduced by cooling that is preferred according to the invention in a continuous process. The temperature of the master batch at the end of the continuous process before cyclically going through steps a) and b) is preferably below 35° C., in particular 25° C. or less.

The premixture prepared in the batch process can be cooled in a continuous process in various ways. A continuous system in which a corresponding continuous process can be carried out comprises a main line in which the different constituents of the composition according to the invention are introduced in a predetermined, defined order via secondary lines. Furthermore, the highly concentrated premixture, which is usually prepared in the batch process, is diluted with a suitable solvent, usually water. Cooling can now take place by the supplied components and the solvent having a lower temperature than that of the premixture. Furthermore, it is also possible for appropriate cooling devices to be attached around the main pipe, in which mixing occurs due to the flow properties. According to the invention, cooling can take place directly or indirectly. Suitable apparatuses (cooling devices) are plate heat exchangers, tube bundle heat exchangers, or double tube heat exchangers with or without a mixing element in the product-side tube.

In order to allow improved mixing, static and/or dynamic mixers can be provided in the main line of the continuous system. If static mixers are provided, these can also act as cooling. For this purpose, the static mixers can comprise a material that can be cooled, such as a metal or a thermally conductive plastic. It is also conceivable for a suitable coolant to flow through the static mixer, as a result of which the mixture is cooled.

The continuous process is characterized in that there is preferably a positive pressure relative to the ambient pressure within the system in which the continuous process takes place. The mixture is guided through a line system. The flow rate of the composition and thus also the pressure in the line system are controlled by means of pumps. Pressure sensors attached to the line system make it possible to control the pressure within the line system via feedback to the pumps. For example, pressure sensors from Endress and Hauser, Germany, can be used. The main line into which the mixture is introduced or the material flow flowing therein, is called the main flow. The other active substances or components of the composition are also metered into this main line. The continuous process under positive pressure also makes it possible to prevent the entry of gas/air into the composition. Preferably, the continuous method is carried out at a pressure of from 0.1 to 6 bar, in particular from 0.5 to 4 bar, which pressure is elevated compared with the ambient pressure.

In this continuous process, all material is metered together in liquid form into the main line and is homogenized by means of a dynamic and/or static mixer in a continuous system. Liquid products within the meaning of the present invention are liquids or solutions of solids in a suitable solvent as well as stable suspensions, dispersions or emulsions.

A substance, e.g. a composition or a mixture, is liquid according to the definition of the invention if it is in the liquid physical state at 25° C. and 1013 mbar. A substance is solid according to the invention if it is in the solid physical state at 25° C. and 1013 mbar.

The term pairs of surfactant/surfactants, phosphonate/phosphonates, anionic surfactant/anionic surfactants, non-ionic surfactant/non-ionic surfactants and similar terms are intended to have the same meaning and to cover both the singular and the plural.

The premixture preferably has a high concentration of surfactant. This is preferably at least one anionic surfactant. The premixture preferably comprises anionic surfactant in a proportion of from 5 wt. % to 40 wt. %, in particular from 8 wt. % to 36 wt. %, particularly preferably from 10 wt. % to 30 wt. %, even more preferably from 20 wt. % to 28 wt. %.

More preferably, the premixture comprises non-ionic surfactant in a proportion of from 1 wt. % to 27 wt. %, in particular from 10 wt. % to 26 wt. %, particularly preferably from 15 wt. % to 25 wt. %.

Anionic surfactants and non-ionic surfactants can be easily incorporated into a suitable solvent in the batch process. This allows the preparation of a premixture having a high concentration of surfactants, which premixture can then be diluted in a continuous process depending on the desired end product. This allows a high degree of flexibility in the preparation of the desired composition. Master batches according to the invention produced in a batch process, in particular premixtures which have at least one surfactant (surfactant mixtures), are usually stable at only elevated temperature, so that the mixture prepared in the batch process preferably has a temperature of above 40° C. and, as long as it has this temperature, is introduced into the continuous process. In this case, it is desirable for the surfactant mixture to be rapidly diluted in the continuous process, since otherwise the surfactants may flocculate. In addition to the surfactants, there may also be flocculation of soaps or phosphonates. In the case of certain surfactants, a slow dilution would result in a mixture of very high viscosity, which could then no longer be processed. Rapid dilution can easily be made possible in the continuous process, since the metering of the mixture relative to the metering of water into the main line is easy to control. It is particularly preferred to use a high-shear mixer (shear rate 1,000 l/s to 10,000 l/s) in the continuous system for mixing, for example what is known as a Pentax mixer. This allows a more flexible preparation of a starting mixture in the batch, since there are now fewer limitations with regard to the batch mixture. This allows more highly concentrated mixtures, which can be flexibly differentiated by dilution in a continuous system. In addition, the solvent content in the batch mixture can be reduced, which is why a smaller batch boiler can be used. This saves costs in terms of investment, cleaning and maintenance. The present invention thus makes it possible to prepare the agents more cheaply and more efficiently.

The term phosphonate is understood here to mean phosphonates which act as complexing agents in the compositions prepared according to the invention. It should be emphasized that complexing agents are an important constituent of compositions according to the invention. This is why it is so advantageous to be able to use large amounts of phosphonates in preparation methods.

In a very particularly preferred embodiment, the premixture prepared in the batch process has a total phosphonate content of from 0.5 wt. % to 8.0 wt. %, preferably from 1.0 wt. % to 5 wt. %, even more preferably from 1.5 wt. % to 3.0 wt. %. In addition to 1-hydroxyethane-1,1-diphosphonic acid, the complexing phosphonates include a number of different compounds such as diethylenetriamine penta(methylene phosphonic acid) (DTPMP). Hydroxy alkane or amino alkane phosphonates are particularly preferred in this application. Among the hydroxy alkane phosphonates, 1-hydroxyethane-1,1-diphosphonate (HEDP) has particular significance as a cobuilder. It is preferably used as a sodium salt, the disodium salt reacting neutral and the tetrasodium salt reacting alkaline (pH 9). Possible preferable aminoalkane phosphonates include ethylenediamine tetramethylene phosphonate (EDTMP), diethylentriamine pentamethylene phosphonate (DTPMP) and the higher homologs thereof. They are preferably used in the form of the neutrally reacting sodium salt, for example as the hexasodium salt of EDTMP or as the hepta- and octa-sodium salt of DTPMP. Of the class of phosphonates, HEDP is preferably used as a builder. The aminoalkane phosphonates additionally have a pronounced heavy-metal-binding power.

Accordingly, it may be preferred, in particular if the compositions also include bleach, to use aminoalkane phosphonates, in particular DTPMP, or to use mixtures of the mentioned phosphonates.

A prepared master batch that is preferred in the context of this application, in particular the premixture, includes one or more phosphonate(s) from the group
a) aminotrimethylene phosphonic acid (ATMP) and/or the salts thereof;
b) ethylenediamine tetra(methylene phosphonic acid) (EDTMP) and/or the salts thereof;
c) diethylenetriamine penta(methylene phosphonic acid) (DTPMP) and/or the salts thereof;
d) 1-hydroxyethane-1,1-diphosphonic acid (HEDP) and/or the salts thereof
e) 2-phosphonobutane-1,2,4-tricarboxylic acid (PBTC) and/or the salts thereof;
f) hexamethylenediamine tetra(methylene phosphonic acid) (HDTMP) and/or the salts thereof;
g) nitrilotri(methylene phosphonic acid) (NTMP) and/or the salts thereof.

Particularly preferred mixtures are those which include 1-hydroxyethane-1,1-diphosphonic acid (HEDP) or diethylnetriaminepenta(methylene phosphonic acid) (DTPMP) as phosphonates. The mixtures according to the invention may, of course, include two or more different phosphonates. Mixtures that are preferred according to the invention are characterized in that the washing or cleaning agent includes at least one complexing agent from the group of phosphonates, preferably 1-hydroxyethane-1,1-diphosphonate, the proportion by weight of the phosphonate with respect to the total weight of the premixture preferably being from 0.1 to 8.0 wt. %, more preferably from 0.2 to 5.0 wt. % and in particular from 0.5 to 3.0 wt. %.

In a further preferred embodiment, the premixture prepared in the batch process has a total fatty acid content of from 3.0 wt. % to 20 wt. %, preferably from 5.0 wt. % to 15 wt. %, even more preferably from 7.0 wt. % to 10 wt. %.

Within the meaning of the present invention, “stable” means that creaming, phase separation, sedimentation, flocculation or specks, clouds, cloudiness, a milky appearance, solidification or color changes are not observed. The mixture (master batch) prepared in the batch process is preferably stable over a period of 1 day or more, in particular 5 days or more or of 1 week or more, preferably of 2 weeks or more and in particular of 3 weeks or more, preferably for 4 weeks or more, when stored at a temperature of 40° C. or above, in particular from 40° C. to 90° C. The master batch, when stored at 40° C., is preferably stable for 2 weeks or longer, in particular 4 weeks. The same definitions of stability apply to the premixture.

The compositions prepared according to the invention are preferably stable over a period of 4 weeks or more, in particular 8 weeks or more, preferably 12 weeks or more. The composition can be stored at room temperature or above, in particular at 20° C. to 40° C. Particularly preferably, the composition is stable when stored at 40° C. for a period of at least 12 weeks.

According to the invention, the compositions can comprise one or more surfactants. These surfactants are selected from the group consisting of anionic, cationic, zwitterionic, non-ionic surfactants and mixtures thereof. If the compositions or the master batch comprise a plurality of surfactants, these surfactants can be, for example, a plurality of different non-ionic surfactants. However, it is also possible for the compositions or the master batch to comprise, for example, both non-ionic and anionic surfactants. This applies analogously to the other surfactants. The compositions and/or the mixture preferably comprise at least one anionic surfactant and at least one non-ionic surfactant. If the premixture comprises one or more surfactants, further surfactants can be added in a continuous process if required.

Anionic surfactants are preferably selected from the group consisting of C₉-C₁₃ alkyl benzene sulfonates, olefin sulfonates, C₁₂-C₁₈ alkane sulfonates, ester sulfonates, alk(en)yl sulfates, fatty alcohol ether sulfates and mixtures thereof. It has been found that these sulfonate and sulfate surfactants are particularly well suited to preparing stable liquid compositions, in particular those having a yield point. Liquid compositions which comprise C₉-C₁₃ alkyl benzene sulfonates and fatty alcohol ether sulfates as the anionic surfactant have particularly good dispersing properties. Surfactants of the sulfonate type that can be used are preferably C₉₋₁₃ alkylbenzene sulfonates, olefin sulfonates, i.e. mixtures of alkene and hydroxyalkane sulfonates, and disulfonates, as obtained, for example, from C₁₂₋₁₈ monoolefins having a terminal or internal double bond by way of sulfonation with gaseous sulfur trioxide and subsequent alkaline or acid hydrolysis of the sulfonation products. C₁₂₋₁₈ alkane sulfonates and the esters of α-sulfofatty acids (ester sulfonates) are also suitable, for example the α-sulfonated methyl esters of hydrogenated coconut, palm kernel or tallow fatty acids.

The alkali salts and in particular the sodium salts of the sulfuric acid half-esters of C₁₂₋₁₈ fatty alcohols, for example from coconut fatty alcohol, tallow fatty alcohol, lauryl alcohol, myristyl alcohol, cetyl alcohol or stearyl alcohol, or of C₁₀-C₂₀ oxo alcohols and the half-esters of secondary alcohols having these chain lengths are preferred as alk(en)yl sulfates. From a washing perspective, C₁₂-C₁₆ alkyl sulfates, C₁₂-C₁₅ alkyl sulfates and C₁₂-C₁₅ alkyl sulfates are preferred. 2,3-alkyl sulfates are also suitable anionic surfactants.

Fatty alcohol ether sulfates, such as the sulfuric acid monoesters of straight-chain or branched C₇₋₂₁ alcohols ethoxylated with 1 to 6 mol ethylene oxide, such as 2-methyl-branched C₉₋₁₁ alcohols having, on average, 3.5 mol ethylene oxide (EO) or C₁₂₋₁₈ fatty alcohols having 1 to 4 EO, are also suitable.

Preferably, at least one, preferably all, of the liquid compositions according to the invention include a mixture of sulfonate and sulfate surfactants. In a particularly preferred embodiment, the liquid composition and/or the mixture prepared in the batch process includes C₉₋₁₃ alkyl benzene sulfonates and fatty alcohol ether sulfates as the anionic surfactant.

In addition to the anionic surfactant, the liquid compositions may also include soaps. Saturated and unsaturated fatty acid soaps are suitable, such as the salts of lauric acid, myristic acid, palmitic acid, stearic acid, (hydrogenated) erucic acid and behenic acid, and in particular soap mixtures derived from natural fatty acids, such as coconut, palm kernel, olive oil or tallow fatty acids.

The anionic surfactants, and the soaps, can be present in the form of the sodium, potassium, magnesium or ammonium salts thereof. The anionic surfactants are preferably present in the form of the sodium salts thereof. Further preferred counterions for the anionic surfactants are also the protonated forms of choline, triethylamine, monoethanolamine or methylethylamine. The compositions may also comprise at least one non-ionic surfactant. The non-ionic surfactant includes alkoxylated fatty alcohols, alkoxylated fatty acid alkyl esters, fatty acid amides, alkoxylated fatty acid amides, polyhydroxy fatty acid amides, alkylphenol polyglycol ethers, amine oxides, alkyl polyglucosides, and mixtures thereof.

Alkoxylated, advantageously ethoxylated, in particular primary alcohols having preferably 8 to 18 C atoms and, on average, 4 to 12 mol ethylene oxide (EO) per mol of alcohol, in which the alcohol functional group can be linear or preferably methyl-branched in the 2 position, or can include linear and methyl-branched functional groups in admixture, as are usually present in oxo alcohol functional groups, are preferably used as a non-ionic surfactant. However, alcohol ethoxylates having linear functional groups of alcohols of native origin having 12 to 18 C atoms, for example of coconut, palm, tallow fatty or oleyl alcohol, and an average of 5 to 8 EO per mole of alcohol are particularly preferred. Preferred ethoxylated alcohols include, for example, C₁₂₋₁₄ alcohols having 4 EO or 7 EO, C₉₋₁₁ alcohols having 7 EO, C₁₃₋₁₅ alcohols having 5 EO, 7 EO or 8 EO, C₁₂₋₁₈ alcohols having 5 EO or 7 EO, and mixtures thereof. The degrees of ethoxylation indicated represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO. Non-ionic surfactants that include EO and PO groups together in the molecule can also be used according to the invention. Furthermore, a mixture of a (more highly) branched ethoxylated fatty alcohol and an unbranched ethoxylated fatty alcohol, such as a mixture of a C₁₆₋₁₈ fatty alcohol having 7 EO and 2-propylheptanol having 7 EO, is also suitable. Particularly preferably, the washing, cleaning, post-treatment or auxiliary washing agent includes a C₁₂₋₁₈ fatty alcohol having 7 EO or a C₁₃₋₁₅ oxo alcohol having 7 EO as the non-ionic surfactant.

The compositions prepared according to the invention further comprise, in the master batch, one or more solvents. These may be water and/or non-aqueous solvents. The mixture preferably includes water as the main solvent. The premixture prepared in the batch process may further comprise non-aqueous solvents. Suitable non-aqueous solvents include monovalent or polyvalent alcohols, alkanol amines or glycol ethers. Preferably, the solvents are selected from ethanol, n-propanol, i-propanol, butanols, glycol, propanediol, butanediol, methylpropanediol, glycerol, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol mono methyl ether, dipropylene glycol mono ethyl ether, methoxytriglycol, ethoxytriglycol, butoxytriglycol, 1-butoxyethoxy-2-propanol, 3-methyl-3-methoxybutanol, propylene-glycol-t-butylether, di-n-octylether, and mixtures of these solvents.

If the composition according to the invention comprises one or more non-aqueous solvents, in particular those having low vapor pressure, such as ethanol or 2-propanol, these are preferably added to the mixture in a continuous process. The continuous process is carried out in a closed system, such that the corresponding solvent cannot evaporate. This reduces and almost eliminates the risk to the environment. According to the invention, it is also possible for water or other suitable solvents, regardless of their vapor pressure, to be introduced in the continuous process.

The compositions according to the invention may further comprise builders and/or alkaline substances. These are particularly preferably added to the premixture or master batch in the batch process. However, it is also possible for these to be added dissolved in a suitable solvent in a continuous process.

Polymeric polycarboxylates are suitable as builders, for example. These are, for example, the alkali metal salts of polyacrylic acid or of polymethacrylic acid, for example those having a relative molecular mass of from 600 to 750,000 g/mol.

Suitable polymers are in particular polyacrylates which preferably have a molecular mass of from 1,000 to 15,000 g/mol. Due to their superior solubility, the short-chain polyacrylates, which have molar masses of from 1,000 to 10,000 g/mol, and particularly preferably from 1,000 to 5,000 g/mol, can in turn be preferred from this group.

In addition, copolymeric polycarboxylates are suitable, in particular those of acrylic acid with methacrylic acid and of acrylic acid or methacrylic acid with maleic acid. To improve water solubility, the polymers can also include allyl sulfonic acids, such as allyloxybenzene sulfonic acid and methallyl sulfonic acid, as monomers.

Suitable builders that can be included in the composition according to the invention are, in particular, silicates, aluminum silicates (in particular zeolites), carbonates, salts of organic di- and polycarboxylic acids, and mixtures of these substances.

Organic builders which may also be present in the composition according to the invention are, for example, the polycarboxylic acids that can be used in the form of the sodium salts thereof, polycarboxylic acids being understood to mean those carboxylic acids that carry more than one acid function. These include, for example, citric acid, adipic acid, succinic acid, glutaric acid, malic acid, tartaric acid, maleic acid, fumaric acid, saccharic acids, aminocarboxylic acids, nitrilotriacetic acid (NTA), methyl glycine diacetic acid (MGDA), and derivatives and mixtures thereof. Preferred salts are the salts of polycarboxylic acids such as citric acid, adipic acid, succinic acid, glutaric acid, tartaric acid, saccharic acids, and mixtures thereof. However, soluble builders, such as citric acid, or acrylic polymers having a molar mass of from 1,000 to 5,000 g/mol are preferably used in the basic formula.

Within the meaning of the present invention, alkaline substances or washing alkalis are chemicals for raising and stabilizing the pH of the composition.

The master batch comprises in particular dyes, perfume compositions, enzymes, perfume capsules, microbeads, opacifying agents, color transfer inhibitors, brighteners, salt solutions, co-surfactants and water or other solvents.

The co-surfactant or the co-surfactants change the micellar structure of the surfactants in the master batch. This effect can be enhanced by one or more electrolytes. This produces a lamellar structure of the surfactants. Corresponding structured washing or cleaning agents having a yield point are described in the prior art, for example in WO 2013/064357 A1. Full reference is made to the content of this application. Within the meaning of the present invention, co-surfactants are amphiphilic molecules having a small, hydrophilic head group. In a binary system with water, these co-surfactants are often only slightly soluble or not soluble at all. Correspondingly, they do not form micelles there either. In the presence of the surfactants of the basic formula, the co-surfactants are incorporated into their associates and thereby change the morphology of these associates. The ball micelles become rod and/or disk micelles. If the total surfactant content is sufficiently high, lamellar phases or structures are formed.

The co-surfactant is preferably selected from the group consisting of alkoxylated C₈-C₁₈ fatty alcohols having a degree of alkoxylation of ≤3, aliphatic C₆-C₁₄ alcohols, aromatic C₆-C₁₄ alcohols, aliphatic C₆-C₁₂ dialcohols, monoglycerides of C₁₂-C₁₈ fatty acids, monoglycerol ethers of C₈-C₁₈ fatty alcohols and mixtures thereof. Other suitable co-surfactants are 1-hexanol, 1-heptanol, 1-octanol, 1,2-octanediol, stearin monoglycerol and mixtures thereof.

Fragrance alcohols such as geraniol, nerol, citronellol, linalool, rhodinol and other terpene alcohols or fragrance aldehydes such as lilial or decanal are also suitable as co-surfactants.

Preferred co-surfactants are C₁₂-C₁₈ fatty alcohols having a degree of alkoxylation of ≤3. These co-surfactants are particularly well incorporated into the preferred associates of anionic and non-ionic surfactant.

Suitable alkoxylated C₁₂-C₁₈ fatty alcohols having a degree of alkoxylation of ≤3 include, for example, i-C₁₃H₂₇O(CH₂CH₂O₂)₂H, i-C₁₃H₂₇O(CH₂CH₂O)₃H, C₁₂-C₁₄ alcohol having 2 EO, C₁₂₋₁₄ alcohol having 3 EO, C₁₃₋₁₅ alcohol having 3 EO, C₁₂₋₁₈ alcohols having 2 EO and C₁₂₋₁₈ alcohols having 3 EO.

Within the meaning of the present invention, an electrolyte is an inorganic salt. Preferred inorganic salts include sodium chloride, potassium chloride, sodium sulfate, sodium carbonate, potassium sulfate, potassium carbonate, sodium hydrogen carbonate, potassium hydrogen carbonate, calcium chloride, magnesium chloride and mixtures thereof. Particularly stable compositions are obtained when sodium chloride or mixtures of sodium chloride and potassium sulfate are used.

The addition of the inorganic salt supports the formation of lamellar structures. In addition, the inorganic salt has an influence on the viscosity, so that the viscosity of the liquid composition can be adjusted using the inorganic salt.

The yield point is preferably generated by metering the co-surfactants and/or one or more electrolytes in the continuous process. This has the advantage that the constituents metered in the continuous process are immediately present in the desired lamellar structure. In particular, the proportion of co-surfactants and/or electrolytes is up to 15 wt. %, preferably up to 10 wt. %, even more preferably up to 5 wt. %, of the final liquid, surfactant-containing composition having a yield point.

The viscosity of the master batch is in particular 1000 mPa·s or less, in particular 100 to 900 mPa·s, preferably 200 to 800 mPa·s, in particular 400 to 700 mPa·s. The viscosity is measured at a temperature of 20° C. using a Brookfield HATDV II viscometer, 20 rpm, spindle 2.

The master batch may also comprise dispersed particles. Within the meaning of the present invention, dispersed particles are not soluble in the solvent of the master batch, but they can be dispersed therein. According to the invention, these dispersed particles can be functional and/or have an aesthetic function. Functional materials affect the effect of the composition, whereas aesthetic materials affect only the appearance or the odor. The dispersed particles are preferably visible particles. This means that the consumer can clearly identify the particles in the compositions with the naked eye and can distinguish these from the other constituents. This preferably means colored particles. Particularly preferably, one or more of the compositions can include a dissolved dye and additionally colored particles which have a color which is a contrasting color to the dissolved dye.

Within the meaning of the present invention, functional dispersed particles may be capsules, abrasives, granules or compounds. The term capsule is understood to mean both aggregates having a core-shell structure and aggregates having a matrix. Core-shell capsules (microcapsules, microbeads) include at least one solid or liquid core which is enclosed by at least one continuous shell, in particular a shell made of polymer(s).

Sensitive, chemical, physically incompatible and volatile components (=active ingredients) of the liquid composition can be enclosed inside the capsules in a storage-stable and transport-stable manner. The capsules can contain, for example, optical brighteners, surfactants, complexing agents, bleaching agents, optical brighteners, bleach activators, bleach catalysts, dyes and fragrances, antioxidants, builders, enzymes, enzyme stabilizers, antimicrobial active ingredients, graying inhibitors, anti-redeposition agents, pH adjusters, electrolytes, detergent boosters, vitamins, proteins, suds suppressors and/or UV absorbers. The capsule fillings can be solids or liquids in the form of solutions or emulsions or suspensions. The dispersed particles can have a density which corresponds to that of the liquid composition. According to the invention, this means that the density of the dispersed particles corresponds to 90% to 10% of the composition. However, it is also possible for the dispersed particles to have a different density. Nevertheless, because of the method according to the invention, it is also possible here to obtain a uniform dispersion of the particles in the composition. They can consist of different materials such as alginates, gelatin, cellulose, agar, waxes or polyethylenes. Particles that do not have a core-shell structure can also have an active ingredient in a matrix made of a matrix-forming material. Such particles are referred to as “speckles.” In these materials, the matrix is formed for example by gelation, polyanion-polycation interactions or polyelectrolyte-metal ion interactions, and this is well known in the prior art, just like the preparation of particles using these matrix-forming materials.

The composition according to the invention is in particular a body care, washing or cleaning agent. Within the meaning of the present invention, body care, washing or cleaning agents include cosmetics, household cleaners, laundry softeners, detergents for laundry, floor care products, all-purpose cleaners, dishwashing detergents for manual and automatic cleaning, heavy-duty detergents, shampoos, shower gels and bubble baths; preferably, it is a washing or cleaning agent.

The method according to the invention thus makes it possible to prepare compositions which are different from one another and which can be filled simultaneously into a common container, the common container having at least two chambers, the first composition being filled into a first chamber, the second composition into a separate second chamber and each additional composition into another separate chamber. The method is preferably characterized in that the master batch cyclically goes through steps a, b and optionally c and optionally further steps such that the at least one first composition and the at least one second composition and optionally the at least one third composition and each further composition are simultaneously filled into separate chambers of the common container.

In a further embodiment, the present invention relates to a multi-chamber bag which is obtained by means of the method according to the invention. In the first chamber there is a first composition which is prepared by a method according to the invention, in the second chamber a second composition prepared according to the invention and optionally third, fourth or further chambers which also include compositions prepared according to the invention.

In a further embodiment, the present invention relates to a method for cleaning textile structures, which method is characterized in that the structure to be cleaned is placed in a drum of a washing machine, a multi-chamber bag according to the invention is placed in the drum of the washing machine and an automated washing program of the washing machine is started.

The method according to the invention thus allows the preparation of multi-chamber bags having different compositions in the respective chambers, it being possible for filling to take place simultaneously. The installation effort is low compared to other options and the use of the system can be optimized.

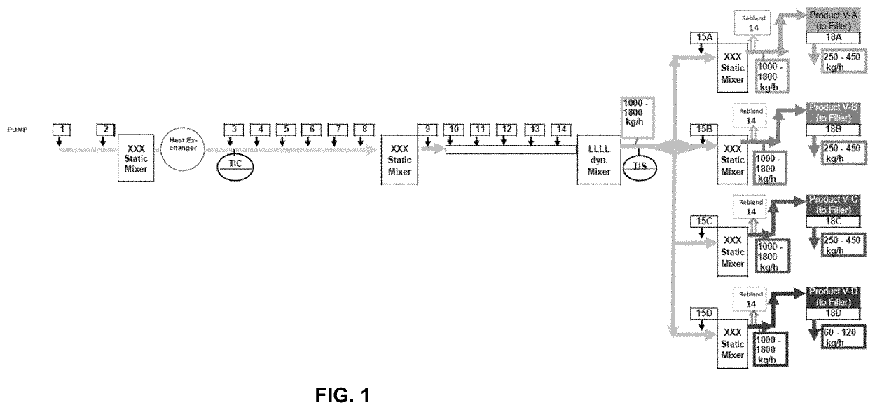

An embodiment that is preferred according to the invention is explained in more detail in the attached FIG. 1. In this embodiment, four different compositions are prepared.

A continuous system is shown in which constituents of the composition according to the invention are metered into the main line via different supply lines. By way of example, the reference signs 1 to 17 denote the supply lines for the following constituents:

| | |
|---|---|
| 1 | solvent (water or non-aqueous solvent) |
| | or master batch |
| 2 | solvent (water or non-aqueous solvent) |
| | or master batch or preservative |
| 3 to 8 | solvent, in particular non-aqueous solvent, |
| | or auxiliaries for adjusting the viscosity |
| | or the pH or preservatives or opacifying |
| | agents or color transfer |
| | inhibitors or brighteners or salt solutions |
| | or surfactants or co-surfactants |
| 9 to 13 | auxiliaries for adjusting the viscosity or |
| | the pH or opacifying agents or color |
| | transfer inhibitors or brighteners or salt |
| | solutions or co-surfactants or perfume |
| | or enzymes |
| 14 | return of mixtures of 15A, 15B, 15C, 15D |
| 15A | first additive |
| 15B | second additive |
| 15C | third additive |
| 15D | fourth additive |

According to the invention, the supply lines can also be used to introduce other or further constituents into the main flow in this order or a different order. The temperature prevailing in the main flow, the number and position of the mixers and the order in which the constituents are added are to be observed by a person skilled in the art. According to the invention, preferably only one material in each case is introduced into the supply line via each of the supply lines. For example, water can be metered via supply line 1, the master batch via supply line 2 and ethanol via supply line 3. Alternatively, it is also possible for ethanol to be metered via supply line 1, water via supply line 2 and the master batch via supply line 3. The same applies to the other supply lines.

The other constituents are in particular:

| | |
|---|---|
| TIC | temperature control |
| TIS | temperature switching point |

Static mixer (XXX static mixer)
Dynamic mixer (LLLL dynamic mixer)
Heat exchanger

In the embodiment shown by way of example in FIG. 1, different supply lines for enzymes (9 to 13) are shown. All of these supply lines are located in the flow direction within the main line in the second half of the system, i.e. are added towards the end of the process. This has the advantage that the mixture is cooled, for example, by a cooler and mixer and optionally the supply of preferably cold water (1, 2, 3, 4, 5, 6, 7, 8) for direct cooling, so that degradation of the enzymes no longer takes place. According to the invention, it is possible to meter enzymes only via one of the supply lines (9, 10, 11, 12, 13). According to the invention, it is also possible to meter enzymes into the main flow via a plurality of supply lines. The same or different enzymes can be metered in via different supply lines. Different enzymes can also be metered via the same lines.

After all the essential constituents have been added, i.e. after inlet 13, the entire flow is directed first in the direction of the inlet 15A after a final mix, where the at least one first additive is added. After a time t1 in which a sufficient amount of the first composition has been produced, the main flow is preferably first diverted and stored separately. This re-blend of the first composition can be reintroduced into the process via supply line 14 when the first composition is prepared again. This period lasts only a few seconds.

The entire flow is then directed in the direction of the inlet 15B, where the second additive is added. This takes place for the time t2. The flow is then diverted in the direction of inlet 15C and then to inlet 15D. The flow is then redirected again to inlet 15A, so that the first, second, third and fourth compositions are obtained cyclically in succession.

What is claimed is:

1. A method for the continuous preparation of at least two different liquid, surfactant-containing compositions, in which method a flow of a master batch which has at least one surfactant and at least one solvent is continuously provided and this master batch cyclically goes through at least the following steps:
a. adding at least one first additive to the flow of the master batch for a first length of time in order to obtain a first composition,
and
b. adding at least one second additive to the flow of the master batch for a second length of time in order to obtain a second composition,
wherein the at least one first additive and the at least one second additive are different from one another and the first and second compositions are stored separately.

2. The method according to claim 1, wherein the master batch cyclically goes through at least the following steps:
a. adding at least one first additive to the flow of the master batch for a first length of time in order to obtain a first composition, and
b. adding at least one second additive to the flow of the master batch for a second length of time in order to obtain a second composition,
c. adding at least one third additive to the flow of the master batch for a third length of time in order to obtain a third composition,
the at least one first additive and the at least one second additive and the at least one third additive being different from one another.

3. The method according to claim 1, wherein the at least one first additive and the at least one second additive and optionally the at least one third and each further additive each have at least one dye, the dyes being different from one another.

4. The method according to claim 1, wherein the at least one first additive and/or the at least one second additive and/or optionally the at least one third and each further additive have one or more enzymes, the enzymes being different from one another.

5. The method according to claim 1, wherein the at least one first additive and/or the at least one second additive and/or optionally the at least one third and each further additive includes at least one optical brightener, an additive comprising either at least one enzyme or at least one optical brightener.

6. The method according to claim 1, wherein the master batch is prepared in a continuous process or a batch process.

7. The method according to claim 1, wherein the master batch is prepared by a premixture comprising at least one surfactant and at least one solvent being prepared in a batch process, to which premixture at least one further component is then added in a continuous process.

8. The method according to claim 7, wherein the proportion of all components of the compositions that are prepared in the batch process is 1 vol. % to 99 vol. % based on the total volume of the master batch.

9. The method according to claim 7, wherein the proportion of all components of the compositions that are prepared in the continuous process is 1 vol. % to 99 vol. % based on the total volume of the master batch.

10. The method according to claim 7, wherein the premixture includes anionic surfactants in a proportion from 5 wt. % to 40 wt. % based on the total weight of the premixture.

11. The method according to claim 7, wherein the premixture includes anionic surfactants in a proportion from 10 wt. % to 30 wt. % based on the total weight of the premixture.

12. The method according to claim 7, wherein the premixture includes non-ionic surfactants in a proportion from 1 wt. % to 27 wt. % based on the total weight of the premixture.

13. The method according to claim 7, wherein the premixture includes non-ionic surfactants in a proportion from 10 wt. % to 26 wt. % based on the total weight of the premixture.

14. The method according to claim 1, wherein the different compositions prepared are filled into a common container which has a separate chamber for each composition.

15. The method according to claim 14, wherein the master batch cyclically goes through steps a., b. and optionally c. such that the at least one first composition and the at least one second composition and optionally the at least one third composition and each further composition are simultaneously filled into separate chambers of the common container.

16. The method according to claim 1, wherein the surfactant-containing compositions are body care agents.

17. The method according to claim 1, wherein the surfactant-containing compositions are washing or cleaning agents.

18. A multi-chamber bag having at least two separate chambers, wherein the first chamber and the second chamber, include the first composition and the second composition, respectively, and each of said first and second compositions is prepared using a method according to claim 1.

19. A method for cleaning textile structures, wherein the structure to be cleaned is placed in a drum of a washing machine, and a multi-chamber bag according to claim 15 is placed in the drum and an automated washing program of the washing machine is started.
